(19) United States
(12) Patent Application Publication
(10) Pub. No.: US 2003/0180927 A1
(43) Pub. Date: Sep. 25, 2003

(54) Yeast protein methyltransferase hsl7p

(76) Inventors: Sidney Pestka, North Caldwell, NJ (US); Jeffry R. Cook, Kendall Park, NJ (US); Terri Goss Kinzy, Bridgewater, NJ (US); Jin-Hyung Lee, Piscataway, NJ (US); David Norris, Princeton, NJ (US); Brian P. Pollack, Highland Park, NJ (US)

Correspondence Address: Ropes & Gray, Patent Group, One International Place, Boston, MA, 02110-2624, US

(21) Appl. No.: 10/239,587

(22) PCT Filed: Mar. 22, 2001
(86) PCT No.: PCT/US01/09087

(51) Int. Cl. A61K 38/51; C12N 1/18; C12N 9/10

(52) U.S. Cl. 435/193; 424/94.5; 435/254.21

(57) ABSTRACT

The present invention relates to yeast protein Hs17p, which is a homologue of Janus kinase binding protein 1, JBP1. Hs17p is a newly characterized protein methyltransferase. The yeast protein Hs17p is a sequence and functional homologue of JBP1 indicating an intricate link between protein methylation and macroscopic changes in yeast morphology.

### BACKGROUND OF THE INVENTION

This application claims priority from U.S. provisional patent application serial No. 60/191,614, filed Mar. 23, 2000.

### GOVERNMENT LICENSING RIGHTS

The experiments in this application were supported in part by United States Public Health Services Grants RO1-CA46465 and 1P30-CA72720 from the National Cancer Institute, RO1 AI36450 and RO1 AI43369 from the National Institute of Allergy and Infectious Diseases, an award from the Milstein Family Foundation, New Jersey Commission on Cancer Research Grant #797777-007, and a National Institute of General Medical Science grant. Further support was provided by NIH grant RO1-GM57058, training grant 2T32AI007403 from the National Institutes of Allergy and Infectious Diseases, and New Jersey Commission on Cancer Research Grant #94-2006-CCR00.

### FIELD OF THE INVENTION

The present invention relates to yeast protein Hs17p, which is a homologue of Janus kinase binding protein 1, JBP1. Hs17p is a newly characterized protein methyltransferase. The yeast protein Hs17p is a sequence and functional homologue of JBP1 indicating an intricate link between protein methylation and macroscopic changes in yeast morphology.

### DESCRIPTION OF THE BACKGROUND

The disclosures referred to herein to illustrate the background of the invention and to provide additional detail with respect to its practice are incorporated herein by reference and, for convenience, are numerically referenced in the following text and respectively grouped in the appended bibliography.

The Jak-Stat pathway plays a crucial role in the signal transduction of many cytokines, growth factors and hormones. Central to this pathway are the Jak family of protein tyrosine kinases. This family includes the mammalian kinases Jak1, Jak2, Jak3 and Tyk2 and the Drosophila melanogaster kinase encoded by the hopscotch(hop) locus. The Jaks are essential for the biological activities mediated by these ligands and defects in this family of kinases have been shown to lead to a number of disease states in both mammals and Drosophila melanogaster.

The role of the Jak kinases in cytokine signal transduction was first shown for the interferons (IFNs). Subsequently, many reports have demonstrated that Jak activation occurs rapidly after ligand stimulation. This activation initiates a cascade of events which includes receptor phosphorylation and recruitment, subsequent phosphorylation and nuclear translocation of members of the Stat (Signal Transducers and Activators of Transcription) family of proteins which then activate cytokine inducible genes. In addition to their enzymatic role, several reports have demonstrated that the Jaks play a structural role in the receptor complex and that the Jaks may have functions in addition to their kinase activity which are important for signaling. For example, introduction of a kinase-inactive mutant of Jak1 into cells that lack this kinase (and are unresponsive to interferon-λ (IFN-λ)) restores partial IFN-λ-induced gene expression. Furthermore, the amino terminus of Tyk2 stabilizes the IFNAR1 chain of the IFN-α receptor complex.

In addition to their interactions with cytokine receptor chains, a large body of evidence has accumulated demonstrating that the Jak kinases interact with other signaling proteins. In particular, Jak2 was reported to interact with SHPTP1, SHPTP2, PP2A, P13K, Yes, Fyn, Shc, Syp, Grb2, the angiotensin II AT1 receptor and the serotonin 5-HT2A receptor (31-44). The ability to interact with such diverse proteins underscores the complex role of Jak2, which is activated by the majority of ligands that utilize the Jak-Stat pathway. While the physiological roles for these interactions have not been characterized, they suggest that the Jaks play a role in other pathways and/or facilitate crosstalk between signaling pathways.

To delineate events which occur downstream of the interferon and other receptors, a two-hybrid screen was used to identify proteins which bind to Jak2. Four Jak2-binding proteins were identified. One, designated Janus kinase binding protein 1, JBP1, (the abbreviations used herein are: JBP1, Janus kinase binding protein 1; AdoMet, S-adenosy-L-methionine; MBP, myelin basic protein) represents the human member of a conserved group of proteins implicated in control of the cell cycle and cell morphology. JBP1 exhibits homology to several other protein methyltransferases in the region to which AdoMet binds. In addition, we reported that JBP1 is a protein methyltransferase capable of methylating histones 2A and 4 and MBP.

The HSL7 (histone synthetic lethal 7) gene of Saccharomyces cerevisiae was originally defined as a gene which is lethal when mutated in combination with histone H3. In addition, Hs17p was found to be a negative regulator of Swe1p and Ste20p function as well as a protein which associates with the septin ring during bud formation. Disruption of HSL7 was reported to result in cell cycle abnormalities and the production of extremely long buds

### BRIEF DESCRIPTION OF THE FIGURES

FIG. 1 illustrates a comparison of JBP1 and Hs17p sequences.

FIG. 2 illustrates the UV crosslinking of [³H]AdoMet to Hs17p. FIG. 2A shows the UV crosslinking of [³H]AdoMet to Hs17p, with details essentially the same as those described previously. FIG. 2B show the in vitro methylation of protein substrates by Hs17p. FIG. 2C shows the inhibition of myelin basic protein in vitro methylation by homocysteine.

FIG. 3 illustrates morphological characteristics of different yeast strains used in this study. FIG. 3A shows wild type (TKY307) yeast. FIG. 3B shows yeast with a disrupted HSL7 gene (SPY101). FIG. 3C shows hs17Δ yeast transformed with pGALFLAGHSL7 (SPY103). FIG. 3D shows hs17Δ yeast expressing JBP1 (SPY104). FIG. 3E shows hs17Δ yeast expressing JBP1-MT (SPY105).

FIG. 4 is a bar graph illustrating the effect of HSL7 gene disruption and complementation on elongated bud phenotype in yeast.

FIG. 5 illustrates the amino acid sequence of Hs17p.

FIG. 6 illustrates the amino acid sequence of JBP1.

### SUMMARY OF THE INVENTION

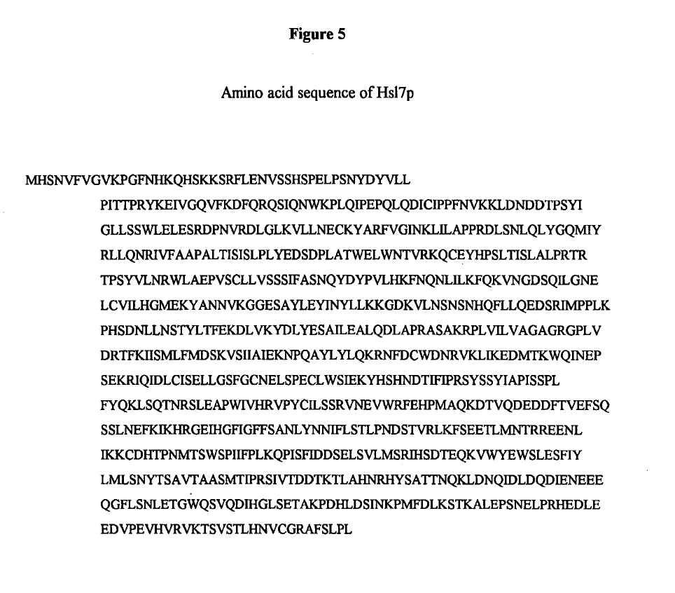

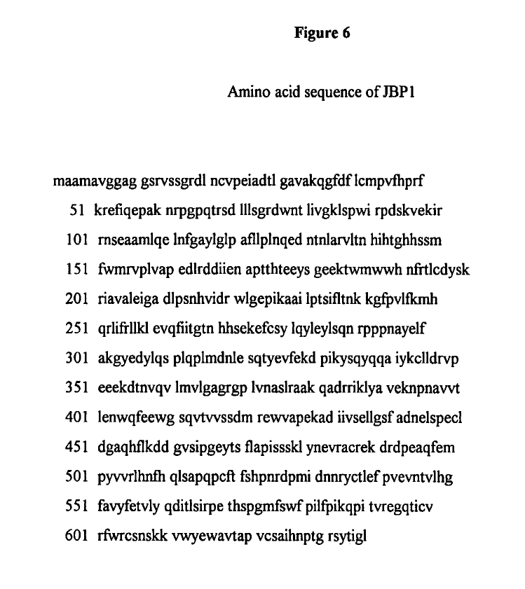

The present invention relates to yeast protein Hs17p, which is a homologue of Janus kinase binding protein 1, JBP1. Hs17p is a newly characterized protein methyltransferase. The yeast protein Hs17p is a sequence and functional homologue of JBP1 indicating an intricate link between protein methylation and macroscopic changes in yeast morphology. Specifically, the present invention pertains to a protein methyltransferase comprising all or a part of the sequence of Hs17p as disclosed in FIG. 5, a homologue to JBP1 comprising all or a part of the sequence disclosed in FIG. 6, a protein methyltransferase expressed by HSL7, a homologue to JBPL expressed by HSL7, and an hs17Δ strain of S. cerevisiae. The present invention further pertains to pharmaceutical compositions for providing interferon therapy to a human comprising a therapeutically effective amount of a protein methyltransferase expressed by HSL7 admixed with a pharmaceutically acceptable vehicle or carrier.

### DETAILED DESCRIPTION OF THE INVENTION

The yeast protein Hs17p is a homologue of Janus kinase binding protein 1, JBP1, a newly characterized protein methyltransferase. Hs17p also is shown to be a methyltransferase. The yeast protein Hs17p is a sequence and functional homologue of JBP1 indicating an intricate link between protein methylation and macroscopic changes in yeast morphology.

The yeast protein Hs17p is a homologue of Janus kinase binding protein 1, JBP1, a newly characterized protein methyltransferase. As disclosed herein for the first time, Hs17p also is shown to be a methyltransferase. It can be crosslinked to [³H]S-adenosylmethionine and exhibits in vitro protein methylation activity. Calf histones H2A and H4 and bovine myelin basic protein were methylated by Hs17p whereas histones H1, H2B, H3, and bovine cytochrome c were not. We demonstrate that JBP1 can complement Saccharomyces cerevisiae with a disrupted HSL7 gene as judged by a reduction of the elongated bud phenotype; and a point mutation in the JBP1 S-adenosylmethionine consensus binding sequence eliminated all complementation by JBP1.

Because of the homology between Hs17p and JBP1, we hypothesized that Hs17p is also a protein methyltransferase. To test this hypothesis, we produced an hs17Δ strain of S. cerevisiae. The phenotype of the hs17Δ strain is characterized by elongated buds. Here we report that Hs17p is a protein methyltransferase and that JBP1 can complement yeast lacking the HSL7 gene. Therefore, we conclude the yeast protein Hs17p is a sequence and functional homologue of JBP1. These data provide evidence for an intricate link between protein methylation and macroscopic changes in yeast morphology.

To identify Jak2-interacting proteins with the yeast two-hybrid system, we cloned a human homologue of the Schizosaccaromyces pombe skb1 protein and the Saccharomyces cerevisiae protein encoded by the HSL7 gene. The skb1 gene was initially identified during a two-hybrid screen for proteins interacting with the shk1 kinase which represents a member of the p21^{cdc42/Rac1}-activated kinase (PAK) family of protein kinases. Recent data suggest that removal of this protein results in cell cycle abnormalities and that the human homologue of this protein can functionally substitute for skb1. In the past no functional motifs or biochemical activities had been identified for skb1 or HSL7, and prior research focused on identifying a biochemical activity for JBP1.

The present invention can be further illustrated by the following examples of preferred embodiments thereof, although it will be understood that these examples are included merely for purposes of illustration and are not intended to limit the scope of the invention unless otherwise specifically indicated.

### EXPERIMENTAL PROCEDURES

Materials. Calf thymus histones were obtained from Roche Molecular Biochemicals (Indianapolis, Ind.); bovine myelin basic protein, cytochrome c, S-adenosylhomocysteine from Sigma (St. Louis, Mo.); [³H]AdoMet (specific activity 55-85 Ci/mmol) from New England Nuclear (Boston, Mass.); and protein A/G PLUS-agarose beads from Santa Cruz Biotechnology, Inc. (Santa Cruz, Calif.).

Cell Growth Conditions. Standard yeast genetics and transformation methods were employed. Yeast were grown in either YEPD or synthetic media (SD/-Trp) with 2% glucose. Induction of genes under control of the GAL1 promoter (pTKB175) was performed in SD/-Trp with 2% galactose.

Plasmid Constructs. Yeast genomic DNA was obtained from Research Genetics, Inc. (Huntsville, Ala.). The HSL7 gene was amplified with 5′ and 3′ primers CTGCAGTACAAAGGGTTCAGTTTG and GTCGACCAGTATATAGTATACAATGC, respectively, and the amplicon digested with Pst I and Sal I, then subcloned into plasmid pTKB175 under control of the GAL1 promoter and containing the TRP1 marker. The R368A mutant of JBP1 and wild type JBP1 cDNAs were constructed in plasmid pcDNA3 as reported. The JBP1 plasmids were digested with BamHI and ApaI and subcloned into pTKB175 producing plasmids pGALJBP1-MT and pGALJBP1-WT, respectively. The Flag-HSL7 construct was produced by amplifying yeast genomic DNA with the 3′ primer defined above plus a 5′ primer (CGCGGATCCGCGATGGACTACAAGGACGACGATGACAAGATGCATAGCAACGTATTTGTTGGT) which encodes a Flag epitope. The amplified DNA was digested with BamHI and SalI and subcloned into plasmid pTKB175 producing the yeast expression vector pGALFLAGHSL7.

Production of hs17Δ Cells. The hs17::URA3 disruption in pBluescript construct (a gift from M. Grunstein) was transformed into haploid wild type yeast (TKY307; Table 1) and transformants were selected with SD/-Ura medium. Disruption of HSL7 was confirmed by Southern blotting.

Complementation Assay. Cells were grown in 2% galactose overnight. An aliquot was used to reinoculate cultures which were grown to mid-log phase. The cells with and without elongated buds were then counted with a hemocytometer. At least 3 fields were examined for each determination. Photographs of yeast strains were made with a Zeiss Axioplan phase contrast microscope.

UV Crosslinking. Crosslinking of [³H]AdoMet to Hs17p and JBP1 was performed as described.

In Vitro Methylation. Immunoprecipitated Flag-Hs17p was used to methylate histones and myelin basic protein with minor modifications of the method previously described. Each reaction contained 10 ig of substrate proteins (histones, MBP and cytochrome c). [³H]AdoMet was added to a final concentration of 55 iCi/ml. The reaction contained 150 mM NaCl, 50 mM Tris.HCl (pH 8.0), 1% NP-40, 0.8 mM PMSF, 3 μg/ml antipain, 10 ig/ml benzamidine (10⁴ kallikrein-inactivating units/ml) plus 1 μg/ml each of leupeptin, chymostatin and pepstatin. The 50 μl reactions were incubated at 30° C. for 30 minutes.

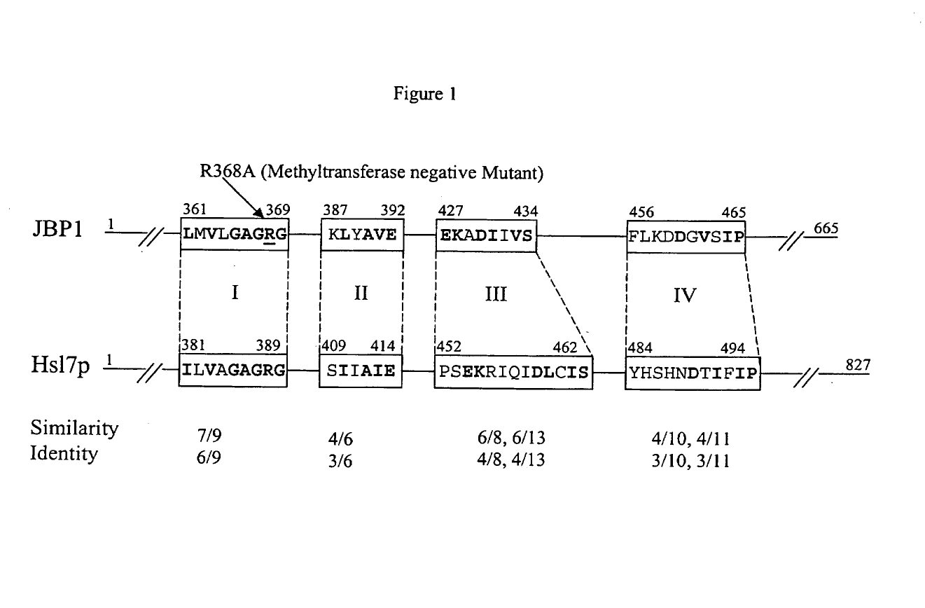

FIG. 1 illustrates a comparison of JBP1 and Hs17p sequences. Identical and similar amino acids are in bold face. R368A indicates the location of the point mutation introduced in subdomain I of the consensus AdoMet binding site. Mutagenesis was performed as described. Similarity/identity numbers were calculated for each of the subdomains (I-IV). For domains III and IV, two percentages were calculated since the domains are of different sizes in HS 17p and JBP1. The first set of percentages indicates similarities and identities based on the length of the HSL7 seauence. The second set of sequences indicates similarities and identities based on the length of the JBP1 sequence.

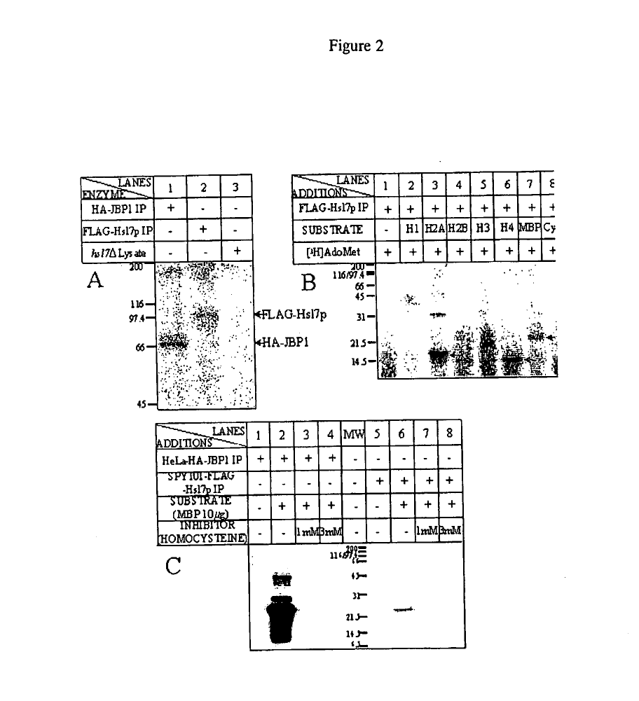

FIG. 2 illustrates the UV crosslinking of [³H]AdoMet to Hs17p. FIG. 2A shows the UV crosslinking of [³H]AdoMet to Hs17p, with details essentially the same as those described previously . After UV crosslinking, the 7.5% gel was dried and exposed to Biomax MR film for 15 days at −70° C. FIG. 2B show the in vitro methylation of protein substrates by Hs17p. Methylation reactions were done as described in “Experimental Procedures.” Each lane contained 10 μg at substrate protein. The position of the labeled bands coincided exactly with the location of the substrate proteins or the Coomassie blue-stained gel (not shown). The dried 15% gel was exposed to Biomax MR film for 21 days at −70° C. FIG. 2C shows the inhibition of myelin basic protein in vitro methylation by homocysteine. Protein methylation reactions were conducted as described in “Experimental Procedures,” except that homocysteine was added to the other reaction components on ice. The reactions were then incubated at 30° C. for 30 minutes. The 15% gel was exposed to Biomax MR film for 14 days at −70° C. Protein sizes were calculated by the migration of broad range protein standards (BioRad).

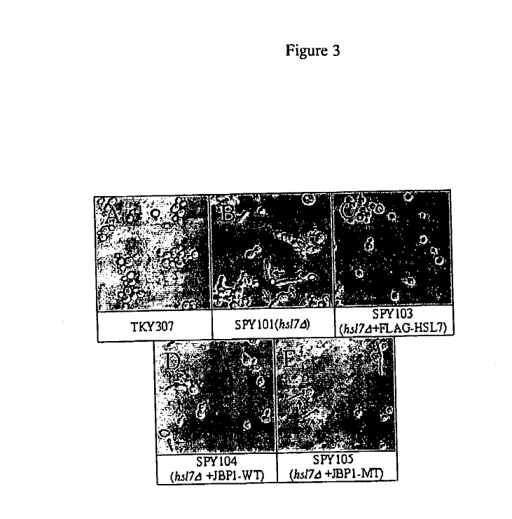

FIG. 3 illustrates morphological characteristics of different yeast strains used in this study. Arrows indicate cells with elongated buds. All cells were grown in media containing 2% galactose to induce the gene under the control of the GAL1 promoter. The elongated bud phenotype was never observed in wild type yeast or in hs17Δ yeast transformed with pGALFLAGHSL7 (SPY103). In the hs17Δ strain, 15 to 20% of the yeast have elongated buds. Complementation with JBP1 reduces the elongated bud phenotype significantly but not completely. FIG. 3A shows wild type (TKY307) yeast. FIG. 3B shows yeast with a disrupted HSL7 gene (SPY101). FIG. 3C shows hs17Δ yeast transformed with pGALFLAGHSL7 (SPY103). FIG. 3D shows hs17Δ yeast expressing JBP1 (SPY104). FIG. 3E shows hs17Δ yeast expressing JBP1-MT (SPY105).

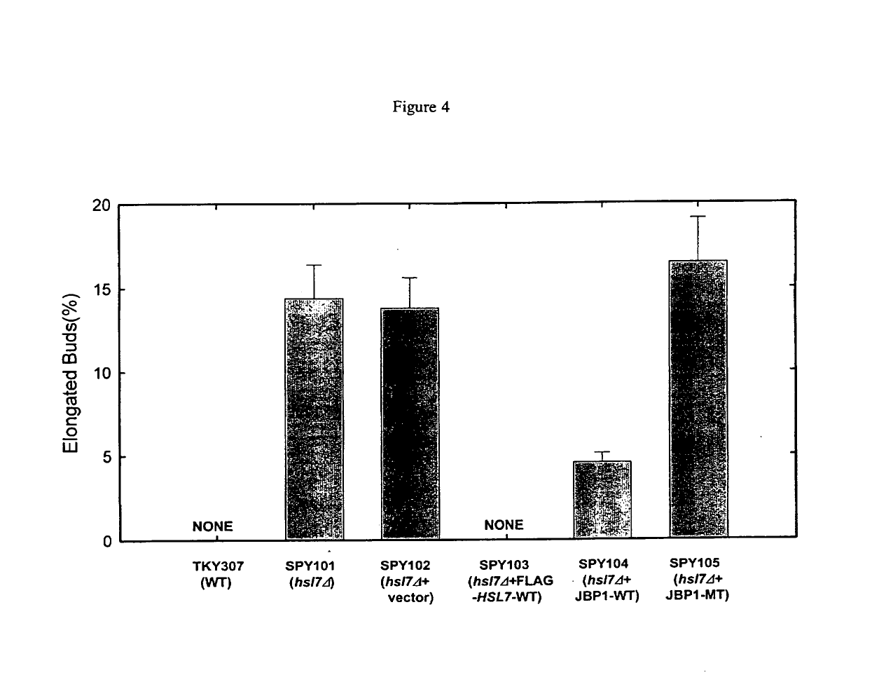

FIG. 4 is a bar graph illustrating the effect of HSL7 gene disruption and complementation on elongated bud phenotype in yeast. Cells were grown in 2% galactose to induce the gene under the control of the GAL1 promoter. At least 3 different fields were counted for each determination. JBP1-WT refers to the wild type JBP1 cDNA; JBP1-MT indicates the mutated (R368A) JBP1 cDNA. Values are ±S.E.M.

FIG. 5 illustrates the amino acid sequence of Hs17p. FIG. 6 illustrates the amino acid sequence of JBP1.

### RESULTS

The human Jak-binding protein, JBP1, was identified in a two-hybrid screen with a 3.33 kb fragment of the Jak2 cDNA as a bait. When the full length JBP1 cDNA was sequenced, it was determined that JBP1 is homologous to a number of other eukaryotic protein methyltransferases, including human ANM1 and ANM2, rat ANM1, and S. cerevisiae Hmt1p. The homology between JBP1 and Hs17p (FIG. 1) suggests that yeast Hs17p is also a protein methyltransferase. There are four conserved subdomains in JBP1 and Hs17p. The first of these regions contains a GxGRG motif which is identical between the human and yeast proteins. This motif is known to be the site at which AdoMet is bound to the protein. FIG. 1 shows the location of a point mutation (R368A) which was introduced in the GxGRG motif in order to produce a JBP1 without methyltransferase activity when histones or myelin basic protein are used as substrates.

To determine whether Hs17p is a methyltransferase, we first asked whether Hs17p can bind [³H]AdoMet. As shown in FIG. 2, JBP1 from HeLa cells was crosslinked to [³H]AdoMet (FIG. 2A, lane 1). The molecular size of this band is 72 kD, which is the size of JBP1. When an anti-flag antibody was used to immunoprecipitate Hs17p from hs17Δ cells transformed pGALFLAGHSL7 (SPY103), a band of 97 kD was observed at the predicted size of Flag-Hs17p (FIG. 2A, lane 2). These results demonstrate that Hs17p binds AdoMet, consistent with its being a methyltransferase. To examine in vitro methylation by Hs17p, calf histones, bovine myelin basic protein and bovine cytochrome c were incubated with immunoprecipitated Hs17p and [³H]AdoMet. FIG. 2B shows that H2A, H4 and myelin basic protein were methylated by Hs17p while H1, H2B, H3 and cytochrome c were not. This pattern of methylation by Hs17p is identical to that observed with human JBP1, indicating that Hs17p and JBP1 are functional as well as structural homologues. The data of FIG. 2C demonstrate that homocysteine, an inhibitor of methyltransferases that use AdoMet as the methyl donor, blocks the methylation of myelin basic protein by both JBP1 and Hs17p.

To investigate whether JBP1 and Hs17p are functional homologues, the HSL7 gene was disrupted in the haploid S. cerevisiae strain TKY307 with an hs17Δ construct which has a 1.14 kb section of the gene replaced with the UR43 gene. The resultant strain, SPY101, was used as a host for other constructs (Table 1).

The hs17 knock-out strain was generated by the homologous recombination as described under “Experimental Procedures.” The plasmids pGALFLAGHSL7, pGALJBP1-WT and pGALJBP1-MT were constructed in the yeast expression vector pTKB175 having a TRP1 marker and are shown in Table 1 illustrating the genotype of strains used in study.

TABLE 1

| | |
|---|---|
| Strain | Genotype |

| | |
|---|---|
| TKY307 | MATα ura3-52 lys2-801 ade2-101 trp1Δ63 |
| | his3Δ200 leu2Δ1 |
| SPY101 | MATα ura3-52 lys2-801 ade2-101 trp1Δ63 |
| | his3Δ200 leu2Δ1 hsl7Δ::URA3 |
| SPY102 | MATα ura3-52 lys2-801 ade2-101 trp1Δ63 |
| | his3Δ200 leu2Δ1 hsl7Δ::URA3 pTRP1 |
| SPY103 | MATα ura3-52 lys2-801 ade2-101 trp1Δ63 |
| | his3Δ200 leu2Δ1 hsl7Δ::URA3 pTRP1 |
| | GALFLAG-HSL7-WT |
| SPY104 | MATα ura3-52 lys2-801 ade2-101 trp1Δ63 |
| | his3Δ200 leu2Δ1 hsl7Δ::URA3 pTRP1 |
| | GALJBP1-WT |
| SPY105 | MATα ura3-52 lys2-801 ade2-101 trp1Δ63 |
| | his3Δ200 leu2Δ1 hsl7Δ::URA3 pTRP1 |
| | GALJBP1-MT |

Vectors pTKB175, pGALFLAGHSL7, pGALJBP1-WT and pGALJBP1-MT were transformed into hs17Δ cells (SPY101) to produce strains SPY102, SPY103, SPY104 and SPY105, respectively. These strains were grown in SD/-Trp with either 2% glucose (uninduced) or 2% galactose (induced). The morphology of these strains was similar to that reported previously: the wild type yeast have small circular or oval buds (FIG. 3A) whereas the hs17Δ cells have elongated buds (FIG. 3B). Complementation of the hs17Δ cells with the HSL7 expression vector yielded cells with a normal phenotype (FIG. 3C). In addition, complementation of the hs17Δ cells with the JBP1-WT expression vector produced cells which were nearly normal (FIG. 3D), but complementation with the mutant JBP1-MT did not (FIG. 3E). Quantitation of the number of cells expressing the elongated bud phenotype in each strain, is shown in FIG. 4. Wild type cells (TKY307) were found to have no elongated buds, whereas 15% of the hs17Δ cells expressed elongated buds (SPY101, FIG. 3B). When hs17Δ cells were transformed with pGALFLAGHSL7 (SPY103, FIG. 3C), the wild type phenotype was completely restored whereas the vector alone had no effect (SPY102, FIG. 4). To determine the extent JBP1 complements its Hs17p homologue, the hs17Δ cells were transformed with the cDNA for human JBP1 expressed under control of the yeast GAL1 promoter. In the presence of 2% galactose, the percentage of cells with elongated buds was reduced by approximately 70% (SPY104, FIG. 4) compared to hs17Δ cells grown in galactose (SPY101, FIG. 4), or compared to hs17Δ+JBP1-WT cells (SPY104) grown in glucose (data not shown).

Since Hs17p and JBP1 may each perform a number of cellular functions, it was important to determine whether the observed phenotypic complementation is due to protein methyltransferase activity of JBP1 or to some other function. JBP1 was therefore mutated (R368A) at the GxGRG motif and the vector expressing the mutant JBP1 was transformed into hs17Δ cells (SPY101). As shown in FIG. 4, hs17Δ+JBP1-MT (SPY105) cells have as many elongated buds as does the hs17Δ strain. This demonstrates that JBP1 is a functional homologue of Hs17p and that the protein methyltransferase activity is required for complementation.

### DISCUSSION

Hs17p is crucial for many functions in yeast. Disruption of HSL7 affects cell morphology, cell cycle progression and sensitivity to chemicals, including calcium, caffeine, calcofluor white, vanadate and verapamil. The fact that Hs17p is a protein methyltransferase and that the mutant JBP1-MT does not complement hs17Δ indicates that some of the phenotypic effects of Hs17p and JBP1 are produced by methylation of target proteins. In our in vitro methylation assays we used histones and myelin basic protein as methyl group acceptors however, the identity of the in vivo substrates for Hs17p remains to be determined.

A number of methyltransferases have been identified in yeast: mRNA cap, rRNA, isoprenylcysteine and tRNA methyltransferases; two protein methyltransferases in S. cerevisiae: Rmt1p (also referred to as Hmt1p or Odp1p;) and Rmt2p. Rmt2p was discovered during a search for yeast proteins containing conserved AdoMet binding motifs, it methylates the δ-nitrogen atom of arginine residues, but its in vivo substrate proteins are not known. Rmt1p, on the other hand, is an arginine methyltransferase which methylates a number of yeast proteins such as Np13p and Hrp1p, which are hnRNPs and poly(A)+ RNA binding proteins. In vitro Rmt1p methylates mammalian hnRNP A1, cytochrome c, histones and myoglobin, but not myelin basic protein. Clearly, Hs17p exhibits different substrate specificity in vitro than Rmt1p. Hs17p methylates myelin basic protein whereas Rmt1p does not; Rmt1p methylates cytochrome c whereas Hs17p does not. These differences imply that Hs17p and Rmt1p play distinct cellular roles.

The phenotypic complementation assays indicate that JBP1 does not completely rescue hs17Δ cells (SPY104). Differences in the yeast lysate proteins methylated by JBP1 and Hs17p (J. -H. Lee, J. R. Cook and S. Pestka, unpublished results) could account for this. The R368A mutation of JBP1 which did not restore normal morphology demonstrated that complementation in hs17Δ yeast absolutely requires methyltransferase activity. Although we have illustrated only four conserved regions in JBP1 and Hs17p (FIG. 1), JBP1 and Hs17p share extensive homology in other regions as well.

The S. pombe homologue of Hs17p is skb1, a protein which is known to interact with the kinase Shk1. A S. pombe skb1 deletion mutant exhibits altered morphology where the wild type cells are more elongated than the mutants and over-expression of skb1 results in hyper-elongated cells. JBP1 was shown to functionally complement skb1 in terms of cell length while Hs17p did not. Nevertheless, the roles of skb1 and Hs17p S. pombe and S. cerevisiae, respectively, are likely to be similar. For example, Skb1 and Hs17p are involved in the ras signaling pathway in S. pombe and in S. cerevisiae; and deletion of both genes produces cells with growth abnormalities.

In S. cerevisiae, Hs17p is a functional component of the MAP kinase pathway where it was shown to compete with Cdc42p for binding to the amino-terminal half of Ste20p. Ste20p is a member of the p65^{PAK} protein kinase family and is involved in several yeast signal transduction pathways. In the haploid mating pathway, Ste20p is a kinase downstream of the Ste12p and Ste3p receptors which bind á-factor and a-factor, respectively. Ste20p, Ste11p, Ste7p, and Ste12p are also required for the switch from an axial to a bipolar mode of budding which results in invasive growth. In diploid cells, nitrogen starvation produces a filamentous type of growth which is mediated through Ste20p and the MAP kinase pathway; Ras2p also enhances this pathway. Hs17p contributes to all of these phenotypes via Ste20p. Hs17p also inhibits the Swe1p kinase that phosphorylates Cdc28, thereby producing changes in the cell cycle. In the Swe1p/Cdc28p morphogenesis checkpoint pathway, Swe1p and Hs11p both associate with Hs17p. Hs17p localizes to septin rings formed at the bud necks of dividing cells where it forms a complex with Hs11p, Swe1p and the septins and is involved in the Cdc28-mediated G2/M cell cycle transition. McMillan et at. reported that Hs11p can phosphorylate Hs17p. While the levels of Hs17p appear to be relatively constant during the cell cycle, Hs11p expression is cell cycle-dependent and so is its phosphorylation of Hs17p. Ultimately Hs17p and Hs11p interact to promote the degradation of Swe1p, possibly by polyubiquitination. Thus, Hs17p is a functional component of both the Swe1p/Cdc28p morphogenesis checkpoint and MAP kinase pathways and may serve as a link between these two pathways

Although methylation of proteins such as the histones was recognized decades ago, a clear function for histone methylation has not been delineated. Recently, the methyltransferase CARM1 was reported to methylate histones H2A and H3 in vitro and enhance the transcription of nuclear receptors, suggesting that it activates transcription through histone methylation. The homologue of Hs17p, JBP1, interacts with all the Janus kinases (Jak1, Jak2, Jak3 and Tyk2), kinases required for signal transduction of interferons, cytokines and growth factors. As described above, Hs17p is intrinsically involved in at least two pathways: the Swe1p/Cdc28p morphogenesis checkpoint; and Ras signaling in the MAP kinase pathway. Furthermore, because Hs17p methylates histones, Hs17p is likely involved in chromatin remodeling and may contribute to the “histone code” that can control downstream events. Our data presented in this report provide evidence that there is an intricate link between protein methylation and yeast morphogenesis and other pathways such as Ras signaling and histone coding; and provide a biochemical basis for understanding the mechanism by which Hs17p modulates these many diverse actions.

Although this invention has been described in connection with specific forms thereof, it should be appreciated that a wide array of equivalents may be substituted for the specific elements shown and described herein without departing from the spirit and scope of this invention as described in the appended claims.

Throughout this application, various publications have been referenced. The disclosures in these publications are incorporated herein by reference in order to more fully describe the state of the art.

Appendium Of References

1. Pollack, B. P., Kotenko, S. V., He, W., Izotova, L., Barnowski, B. L., and Pestka, S. (1999) J. Biol. Chem. 274, 31531-31542

2. Ma, X. -J., Lu, Q., and Grunstein, M. (1996) Genes Dev. 10, 1327-1340

3. Gilbreth, M., Yang, P., Wang, D., Frost, J., Polverino, A., Cobb, M. H., and Marcus, S. (1996) Proc. Natl. Acad. Sci. USA 93, 13802-13807

4. Fujita, A., Tonouchi, A., Hiroko, T., Inose, F., Nagashima, T., Satoh, R., and Tanaka, S. (1999) Proc. Natl. Acad. Sci. USA 96, 8522-8527

5. Shulewitz, M. J., Inouye, C. J., and Thorner, J. (1999) Mol. Cell Biol. 19, 7123-7137

6. Sherman, F., Fink, G. R., and Hicks, J. B. (1986) Methods in Yeast Genetics: A Laboratory Manual, Cold Spring Harbor Laboratory, Cold Spring Harbor, N.Y.

7. Ito, H., Fukuda, Y., Murata, K., and Kimura, A. (1983) J. Bacteriol. 153, 163-168

8. Carr-Schmid, A., Valente, L., Loik, V. I., Williams, T., Starita, L. M., and Kinzy, T. G. (1999) Mol. Cell. Biol. 19, 5257-5266

9. Niewmierzycka, A., and Clarke, S. (1999) J. Biol. Chem. 274, 814-824

10. Cheng, X., Kumar, S., Posfai, J., Pflugarth, J. W., and Roberts, R. J. (1993) Cell 74, 299-307

11. Kucharczyk, R., Gromadka, R., Migdalski, A., Slonimski, P. P., and Rytka, J. (1999) Yeast 15, 987-1000

12. Mao, X., Schwer, B., and Shuman, S. (1995) Mol. Cell. Biol. 15, 4167-4174

13. Lafontaine, D., Delcour, J., Glasser, A. L., Desgres, J., and Vandenhaute, J. (1994) J. Mol. Biol. 241, 492-497

14. Hrycyna, C. A., Sapperstein, S. K., Clarke, S., and Michaelis, S. (1991) EMBO J. 10, 1699-1709

15. Sirum-Connolly, K., and Mason, T. L. (1993) Science 262, 1886-1889

16. Gary, J. D., Lin, W. -J., Yang, M. C., Herschman, H. R., and Clarke, S. (1996) J. Biol. Chem. 271, 12585-12594

17. Henry, M. F., and Silver, P. A. (1996) Mol. Cell Biol. 16, 3668-3678

18. Shen, E. C., Henry, M. F., Weiss, V. H., Valentini, S. R., Silver, P. A., and Lee, M. S. (1998) Genes Dev. 12, 679-691

19. McBride, A. E., Weiss, V. H., Kim, H. K., Hogle, J. M., and Silver, P. A. (2000) J. Biol. Chem. 275, 3128-3136

20. Gilbreth, M., Yang, P., Bartholomeusz, G., Pimental, R. A., Kansra, S., Gadiraju, R., and Marcus, S. (1998) Proc. Natl. Acad. Sci. USA 95, 14781-14786

21. Leberer, E., Dignard, D., Harcus, D., Thomas, D. Y., and Whiteway, M. (1992) EMBO J. 11, 4815-4824

22. Roberts, R. L., and Fink, G. R. (1994) Genes Dev. 8, 2974-85

23. Gimeno, C. J., Ljungdahl, P. O., Styles, C. A., and Fink, G. R. (1992) Cell 6, 1077-1090

24. Mosch, H. U., Roberts, R. L., Fink, G. R. (1996) Proc. Natl. Acad. Sci. USA 93, 5352-5356

25. McMillan, J. N., Longtime, M. S., Sia, R. A. L., Theesfeld, C. L., Bardes, E. S. G., Pringle, J. R., and Lew, D. J. (1999) Mol. Cell. Biol. 19, 6929-6939

26. Chen, D., Ma, H., Hong, H., Koh, S. S., Huang, S. -M., Schurter, B. T., Aswad, D. W., and Stallcup. M. R. (1999) Science 284, 2174-2177

27. Strahl, B. D., and C. D. Allis, C. D. (2000) Nature 403, 41-45

The invention being thus described, it will be obvious that the same may be varied in many ways. Such variations are not to be regarded as a departure from the spirit and scope of the invention and all such modifications are intended to be included within the scope of the following claims.

We claim:

1. A protein methyltransferase comprising all or a part of the sequence of Hs17p as disclosed in FIG. 5.

2. A homologue to JBP1 comprising all or a part of the sequence disclosed in FIG. 6.

3. A protein methyltransferase expressed by HSL7.

4. A homologue to JBP1 expressed by HSL7.

5. An hs17Δ strain of S. cerevisiae.

6. A pharmaceutical composition for providing interferon therapy to a human comprising a therapeutically effective amount of a protein methyltransferase expressed by HSL7 admixed with a pharmaceutically acceptable vehicle or carrier.
